(19) United States
(12) Patent Application Publication
(10) Pub. No.: US 2024/0176934 A1
(43) Pub. Date: May 30, 2024

(54) DESIGN PROCESS FOR IMPLANT OPTIMIZATION AND MANUFACTURING

(71) Applicant: Zimmer, Inc., Warsaw, IN (US)

(72) Inventors: Ravikumar Varadarajan, Warsaw, IN (US); William Paul Rodgers, III, Warsaw, IN (US)

(21) Appl. No.: 18/508,022

(22) Filed: Nov. 13, 2023

Related U.S. Application Data
(60) Provisional application No. 63/428,409, filed on Nov. 28, 2022.

(51) Int. Cl. G06F 30/27; A61F 2/40; G06F 30/10

(52) U.S. Cl. CPC G06F 30/27; A61F 2/40; G06F 30/10; A61F 2002/30952

(57) ABSTRACT

A method for manufacturing an implant can include receiving at a computing device, patient specific data related to a bone of a patient and implant data related to an implant for the patient, and determining, by the computing device, optimal design parameters for the implant for the patient using the patient specific data and a machine learning model. The method can also include exporting, by the computing device, the optimal design parameters to a manufacturing device of systems for manufacturing the implant. The optimal design parameters can provide for a non-solid implant construction in regions of lower stress within the bone of the patient and a solid implant construction in regions for higher stress within the bone of the patient to bring stress distributions closer to those experienced in a non-implanted bone to, in turn, reduce the likelihood of bone resorption and peri-prosthetic fractures after implantation of the implant.

### CLAIM OF PRIORITY

This application claims the benefit of U.S. Provisional Patent Application Ser. No. 63/428,409, filed on Nov. 28, 2022, the benefit of priority of which is claimed hereby, and which is incorporated by reference herein in its entirety.

### FIELD OF THE DISCLOSURE

The present disclosure relates generally to surgical implants for use in joint replacements and the methods of design thereof. More specifically, the present disclosure relates to design processes incorporating artificial intelligence (AI) in conjunction with simulations or modeling to manufacture standard-line and patient-specific implants.

### BACKGROUND

The shoulder (e.g., glenohumeral) joint is the most mobile joint in the human body. In a healthy shoulder joint, the humeral head of the humerus articulates within the glenoid cavity of the scapula, which, together various soft tissues, allows the shoulder joint to articulate through a wide range of motion. However, through injury or disease, degradation of humeral or glenoid bone, or various soft tissues, often leads to corrective surgery to help restore joint functionality, such as in the form of a total shoulder arthroplasty or a reverse shoulder arthroplasty. Both total and reverse shoulder replacement surgeries involve the implantation of a prosthetic shoulder joint that is matched with the bio-kinematics of a patient.

Prosthetic shoulder joints include a humeral implant adapted for fixation to the humerus and a glenoid implant adapted for fixation to glenoid bone within the glenoid cavity of the scapula. In a total shoulder arthroplasty, the humeral implant generally includes a stem for insertion into the humeral medullary canal and a prosthetic humeral head secured thereto for replacing the natural humeral head; and the glenoid implant generally includes a baseplate adapted for fixation to glenoid bone and a prosthetic cup secured thereto to provide an articular surface for the prosthetic humeral head. In a reverse shoulder arthroplasty, the baseplate of the glenoid implant includes a spherical component, often called a glenosphere, and the stem of the humeral implant includes the prosthetic cup to provide an articular surface for the spherical component.

### SUMMARY

The present disclosure relates to design processes incorporating artificial intelligence (AI) and simulations or modeling to manufacture standard-line and patient-specific shoulder implants. The design process can optimize implant parameters to reduce likelihood of post-operative complications.

Patient bone and implant data are input into a machine learning model. The model simulates loading conditions and bone-implant interface to optimize design parameters like lattice structures and porous coatings. Lattice structures in low stress regions allow flexibility while solid construction in high stress regions provides rigidity. This matches implant stiffness to bone stiffness, reducing stress shielding.

Optimized shoulder implants better match patient bone strength and biomechanics. Flexible, non-solid construction in the implant reduces bone resorption and peri-prosthetic fractures. Patient outcomes and recovery are improved.

Some key benefits of the proposed implant design technique can include:
Reduced likelihood of bone resorption and peri-prosthetic fractures by optimizing the implant's flexibility to match the patient's bone stiffness, which can also reduce stress shielding.
Improved post-operative healing by promoting bone ingrowth into porous coatings and lattice structures.
Lighter weight implants that cause less patient discomfort.
Ability to account for patient-specific factors like bone quality and biomechanics through machine learning and simulation.
Automated design optimization through machine learning, finite element analysis, and musculoskeletal modeling.
Flexibility to manufacture both standard and patient-specific implants.
Capability to reinforce high-stress regions with solid construction while allowing flexibility with lattices in low-stress regions.
   Overall, the proposed process allows for automated, customized implant design that is optimized for each patient's unique anatomy and needs. This improves surgical outcomes.

The full extent of the techniques discussed herein are described below with reference to the figures. The above is provided merely as a brief, non-limiting, summary of the disclosure.

### BRIEF DESCRIPTION OF THE FIGURES

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 illustrates a first example surgical site of a patient, in accordance with at least one embodiment of this disclosure.

FIG. 2 illustrates a second example surgical site of a patient, in accordance with at least one embodiment of this disclosure.

FIG. 3A illustrates an example glenoid of a scapula, in accordance with at least one embodiment of this disclosure.

FIG. 3B illustrates an example glenoid with an example second implant affixed thereto, in accordance with at least one embodiment of this disclosure.

FIGS. 4A, 4B, 4C, and 4D each illustrate an example second implant, in accordance with at least one embodiment of this disclosure.

FIGS. 5A, 5B, 5C, and 5D each illustrate a cross-section of an example stem, in accordance with at least one embodiment of this disclosure.

FIG. 6 illustrates an example lattice structure, in accordance with at least one embodiment of this disclosure.

FIGS. 7A & 7B each illustrate a cross-section of an example baseplate, in accordance with at least one embodiment of this disclosure.

FIG. 8 illustrates an example method for manufacturing an implant, in accordance with at least one embodiment of this disclosure.

FIG. 9 illustrates an example method of designing prosthetic implant designs, in accordance with at least one example of this disclosure.

FIG. 10 illustrates an example table of inputs, in accordance with at least one embodiment of this disclosure.

FIG. 11 illustrates an example table of outputs, in accordance with at least one example of this disclosure.

FIG. 12 illustrates an example schematic of computing device, in accordance with at least one example of this disclosure.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

During a shoulder replacement surgery (e.g., a shoulder arthroplasty), implantation of the humeral implant into the humerus first involves a resection of the articular portion thereof (e.g., the humeral head) to remove diseased or damaged bone and expose the medullary canal. The medullary canal, or other portions of the humerus, can then be reamed or rasped to prepare the humerus to receive the stem of the humeral implant. Implantation of the glenoid implant first involves shaping a glenoid surface within the glenoid cavity to prepare the scapula to receive or otherwise engage the baseplate of the glenoid implant. Traditionally, both the stems of humeral implants and the baseplates of glenoid implants have been constructed from solid metallic materials. As can be appreciated, once implanted in a bone, a solid metallic construction can result in a substantial difference in stiffness between the implant and the bone.

This can, in turn, shield bone located near (e.g., peri-prosthetic bone), or in contact with, the implant from the stress forces generated during normal joint movement. Stress-shielding can increase the likelihood of peri-prosthetic bone resorption (e.g., death or breakdown of bone in contact with or located near an implant) or peri-prosthetic bone fractures. For example, during some joint movements, stresses in an implanted bone can be much lower than optimal near the implant, which will lead to bone resorption according to Wolff's law, or during other joint movements, stresses in the bone can be higher than optimal near the implant, which can lead to peri-prosthetic fractures.

Bone resorption can weaken the fixation or engagement between the bone and the implant, which can cause joint instability, and peri-prosthetic fractures can require complex revision surgeries where clinical success rates are often significantly lower than those in initial or primary joint replacement surgeries. Moreover, some patients can have degenerative bone conditions, such as osteoporosis, that can decrease the strength of natural bone and thereby increase a difference in stiffness between a patient's bone and a solid metallic implant, which can, in turn, further increase the likelihood of peri-prosthetic fractures. Additionally, solid metallic implants can be significantly heavier and denser, relative to the weight and density of natural bone, which can cause discomfort for patients, and, in some arthroplasties, make securing the implant to a bone a challenging operation.

To help address the above issues, among others, the present disclosure can provide a design process usable to optimize various implants, such as, but not limited to, humeral or glenoid implants, to enable such implants to reduce the likelihood of post-operative bone resorption or peri-prosthetic bone fractures in implanted bones. For example, the design process of the present disclosure can be used to optimize and create implants having a lattice structure, or a non-solid construction, in regions of lower stress within an implanted bone and a solid structure, or a solid construction, in regions of higher stress, to bring stress forces or stress distributions within the implanted bone closer to those experienced in a natural or non-implanted bone. In this way, the design process of the present disclosure can significantly reduce the stress-shielding caused by solid implants, and in, turn, reduce the likelihood of bone resorption and peri-prosthetic fractures associated therewith.

While the above description is generally discussed with respect to humeral or glenoid implants in the context of shoulder arthroplasties, it to be appreciated that design process of the present disclosure, and any of its associated benefits, is also applicable to a wide variety of other implants for implantation within, or otherwise engaging, various bones within the human body, such as but not limited to, a femoral or acetabular implants in the context of hip or knee arthroplasties, ulnar implants in the context of elbow replacements, or intramedullary nails or bone plates for use in various fracture correction surgeries. The above discussion is intended to provide an overview of subject matter of the present patent application. It is not intended to provide an exclusive or exhaustive explanation of the invention. The description below is included to provide further information about the present patent application.

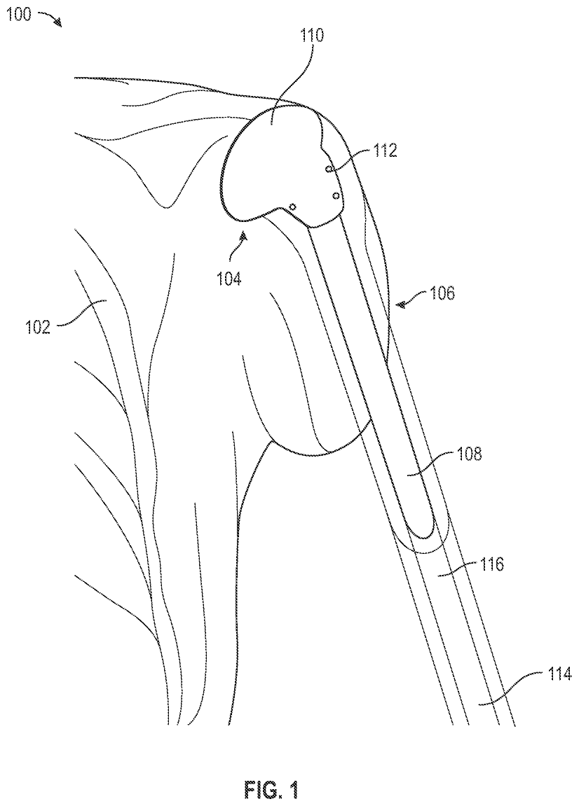

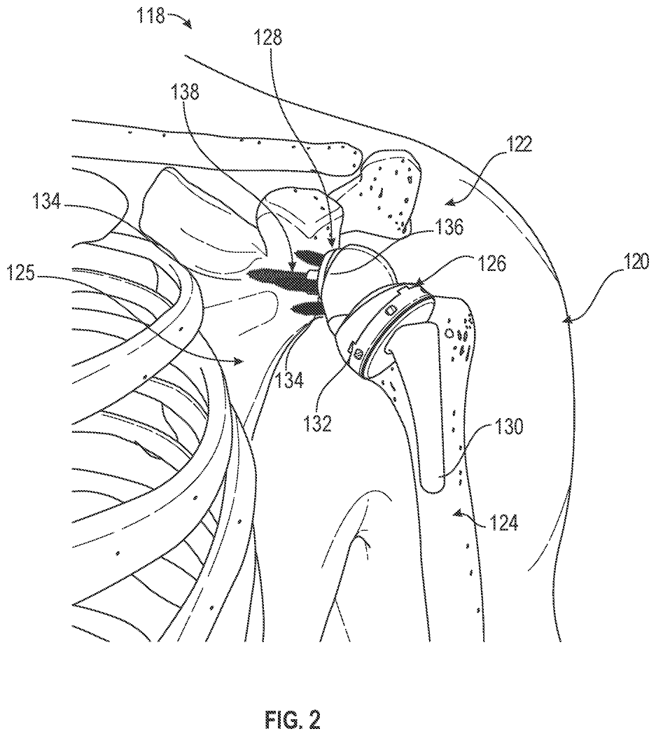

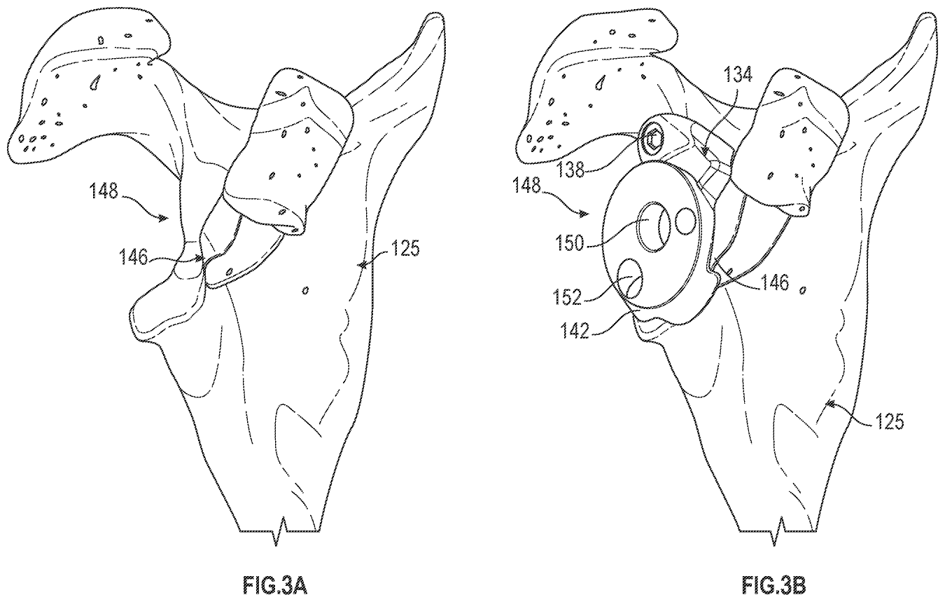

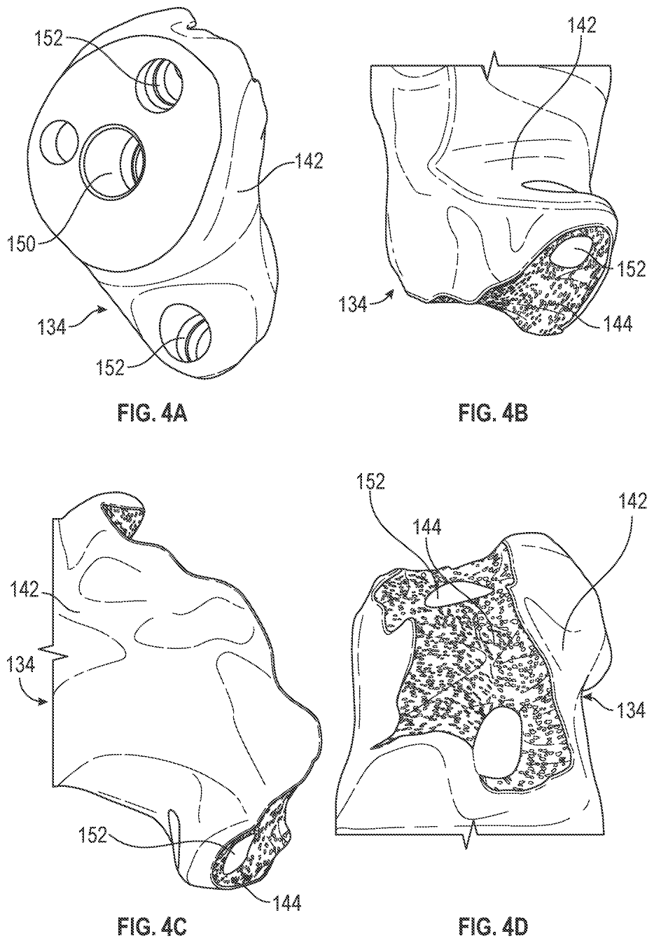

FIG. 1 illustrates a surgical site 100 of a patient 102, in accordance with at least one embodiment of this disclosure. FIG. 2 illustrates an additional example surgical site 118 of a patient 120, in accordance with at least one embodiment of this disclosure. FIG. 3A illustrates an example second bone 125, in accordance with at least one embodiment of this disclosure. FIG. 3B illustrates an example second bone 125 with an example baseplate 134 affixed thereto, in accordance with at least one embodiment of this disclosure. FIGS. 4A-4D each illustrate an example baseplate 134, in accordance with at least one embodiment of this disclosure. FIGS. 3A-4D are discussed below concurrently. FIGS. 1-4D are discussed below concurrently.

With respect to FIG. 1, the surgical site 100 (FIG. 1) can include, or be located proximally to, a joint 104 (FIG. 1) including a bone 114 (FIG. 1). The joint 104 can be generally representative of various joints in the human body in need of replacement. In some examples, the joint 104 can be a shoulder joint (e.g., the glenohumeral joint) and the bone 114 can be a humerus. In other examples, the surgical site 100 can be representative of other surgical sites, such as including, but not limited to, hip, knee, or elbow joints, or an access incision made to access a fractured bone. The joint 104 can be at least partially replaced with an implant 106 (FIG. 1). The implant 106 can be generally representative of a stemmed humeral implant adapted for use in a total shoulder replacement procedure. For example, the implant 106 can include a stem 108 (FIG. 1), an articulation component 110 (FIG. 1), and one or more fixation components 112 (FIG. 1). The articulation component 110 can be, for example, a prosthetic humeral head or spherical component securable to the stem 108 for articulating against a prosthetic cup, such as secured within a glenoid cavity of the patient 102.

Similarly, with respect to FIG. 2, the surgical site 118 (FIG. 2) of the patient 120 can include, or can be located proximally to, a joint 122 (FIG. 2) including a first bone 124 (FIG. 2) and a second bone 125 (FIGS. 2-3B). The joint 122 can be generally representative of various joints in the human body in need of replacement. In some examples, the joint 122 can be a shoulder joint (e.g., the glenohumeral joint), the first bone 124 can be a humerus, and the second bone 125 can be a scapula. In other examples, the surgical site 118 can be representative of other surgical sites, such as including, but not limited to, hip, knee, or elbow joints, or an access incision made to access a fractured bone. The joint 122 can be at least partially replaced with a first implant 126 (FIG. 2) and a second implant 128 (FIG. 2).

The first implant 126 can be generally representative of a stemmed humeral implant adapted for use in a reverse shoulder replacement procedure. For example, the first implant 126 can include a stem 130 (FIG. 2) and an articulation component 132 (FIG. 2). The articulation component 132 can be, for example, a prosthetic humeral cup securable to the stem 130 for articulating against a prosthetic humeral or glenosphere. The second implant 128 can be generally representative of a glenoid implant adapted for use in a reverse shoulder replacement procedure. For example, the second implant 128 can include a baseplate 134 (FIGS. 2 & 3B-4D), a glenosphere 136 (FIG. 2), and a plurality of fixation components 138 (FIGS. 2 & 3B). The baseplate 134 can be representative of a variety of bases or baseplates adapted for fixation to bone within a glenoid cavity 148 of the second bone 125 of the patient 120.

In some examples, such as in shoulder arthroplasties involving patients with severely deficient rotator cuffs or extensive scapular bone loss, the baseplate 134 (FIGS. 3B-4D) can be an augmented patient-specific glenoid implant. For example, the baseplate 134 can include an exterior portion 142 (FIGS. 3B-4D) defining an outer contacting surface 144 (FIGS. 4B-4D) that is patient-specific. For example, the outer contacting surface 144 can be sized and shaped to conform to, and make substantial surface contact with, a glenoid surface 146 (FIGS. 3A-3B) defined by the second bone 125 (FIGS. 3A-3B). The glenoid surface 146 can represent one or more surfaces located within, or defining, a glenoid cavity 148 (FIG. 3A-3B) of the second bone 125.

In such examples, the exterior portion 142 and the outer contacting surface 144 can each be sized and shaped based on a three-dimensional image or model of a shoulder joint of a patient, such as the joint 122 (FIG. 2) of the patient 120 (FIG. 2). In some examples, the baseplate 134 can be representative of a custom glenoid baseplate from the Comprehensive® Vault Reconstruction System available from Zimmer Biomet Inc. of Warsaw, Indiana. The baseplate 134 can also include a socket 150 and a plurality of bores 152. The socket 150 and the plurality of bores 152 can each adapted to receive, and thereby guide, the plurality of fixation elements into the second bone 125. The articulation component 132 can be a glenosphere sized and shaped to articulate against, or within, the articulation component 132 (e.g., a cup) secured to the stem 130 first implant 126. The glenosphere 136 can be securable to the baseplate 134 for articulating against the articulation component 132 secured to the stem 130.

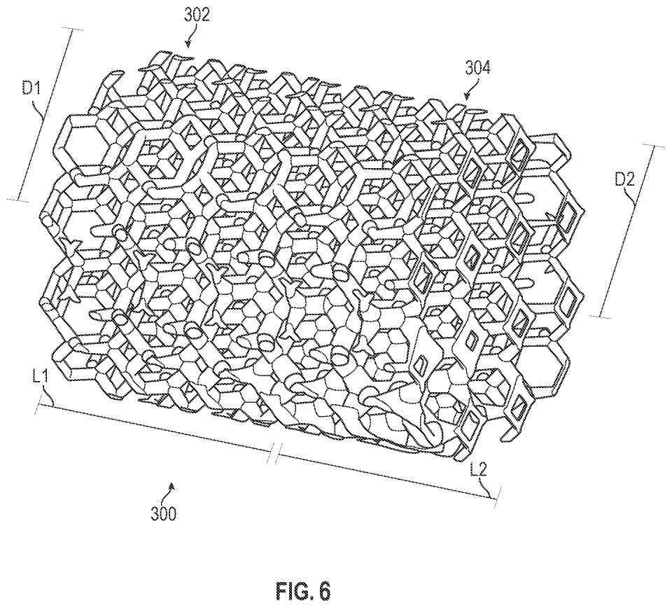

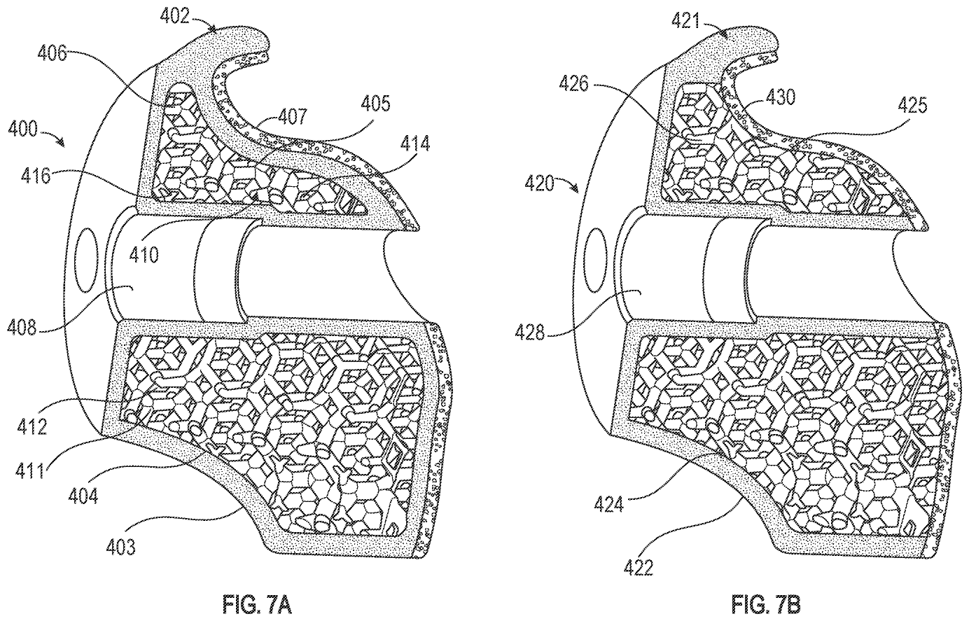

In preparation for a shoulder arthroplasty, various implant parameters of the stem 108 (FIG. 1), or the stem 130 (FIG. 2) and the baseplate 134 (FIGS. 2-4D) can first be optimized using the design process of the present disclosure, which is discussed in detail with respect to FIGS. 8-11 below; to enable the stem 108 (FIG. 1), or the stem 130 and the baseplate 134, to reduce the risk of post-operative bone resorption and peri-prosthetic fracture within the bone 114, or the first bone 124 and the second bone 125. For example, the design process of the present disclosure can be used to design and manufacture the stem 108, or the stem 130 and the baseplate 134, such as by, among others, incorporating lattice structures thereinto, such as shown in FIGS. 4A-4D and FIGS. 7A-7B, to enable the stem 108 or the stem 130 to flex with the first bone 124, and the baseplate 134 to flex with the second bone 125, in regions of lower stress, while also resisting flexing or deformation in regions of higher stress.

During the shoulder arthroplasty, a surgeon can prepare the bone 114 to receive the implant 106 (FIG. 1), or the first bone 124 to receive the first implant 126 (FIG. 2) and the second bone 125 to receive the second implant 128 (FIG. 2). For example, the surgeon can resect a humeral head, or other portions of the bone 114 or the first bone 124, and ream, or otherwise shape, a medullary canal of the bone 114 or the first bone 124, such as to define a cavity 116 (FIG. 1) therein. The cavity 116 can generally be sized and shaped to receive the stem 108 or the stem 130, other portions of the implant 106 or the first implant 126. After preparation of the bone 114, the stem 108 can be inserted into the cavity 116, such as by impacting the stem 108 or the stem 130 into the cavity 116 using a mallet or a hammer, and affixed it therein, such as, but not limited to, via a press fit therebetween or surgical cement.

Next, the articulation component 110 or the articulation component 132 can be secured to, the stem 108 or the stem 130, respectively, such as to replace a natural head portion, or a humeral head, of the bone 114 or the first bone 124, respectively. The surgeon can then prepare the second bone 125 to receive the second implant 128. For example, the surgeon can ream, drill, or otherwise modify various bone surfaces within the glenoid cavity 148 (FIGS. 3A-3B), such as the glenoid surface 146 (FIGS. 3A-3B), to receive or otherwise contact and engage portions of the baseplate 134, or other portions of the second implant 128. After preparation of the second bone 125, the baseplate 134 can be affixed or secured thereto, such as by driving the plurality of fixation components 138 through the plurality of bores 152 (FIGS. FIGS. 3B-4D) into the second bone 125 using a surgical drill or screwdriver. The glenosphere 136, or a cup adapted to articulate against the articulation component 110, can then be secured to the baseplate 134, such as through a tapered or press-fit between one or more portions thereof and a socket 150 extending into the exterior portion 142.

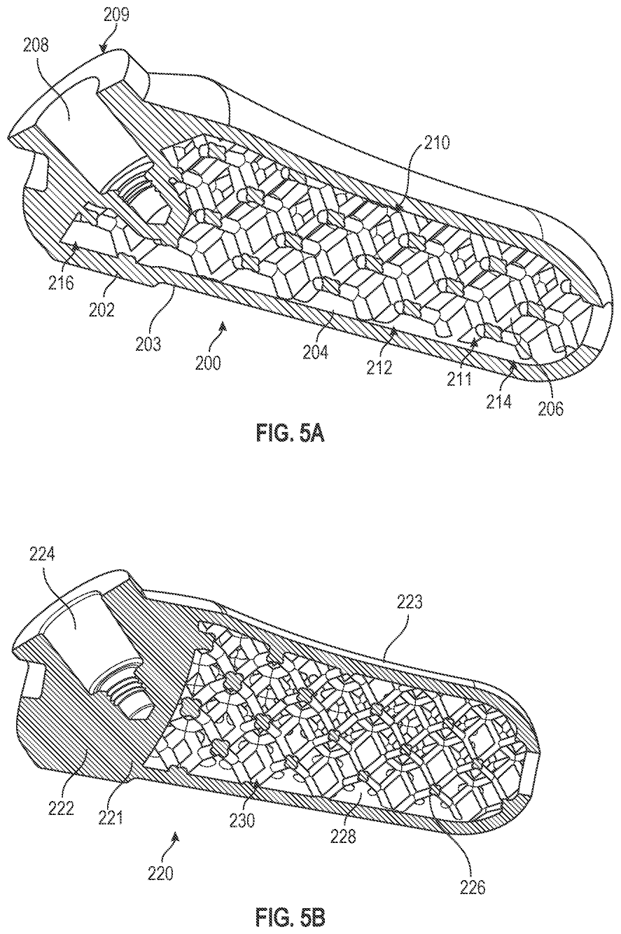

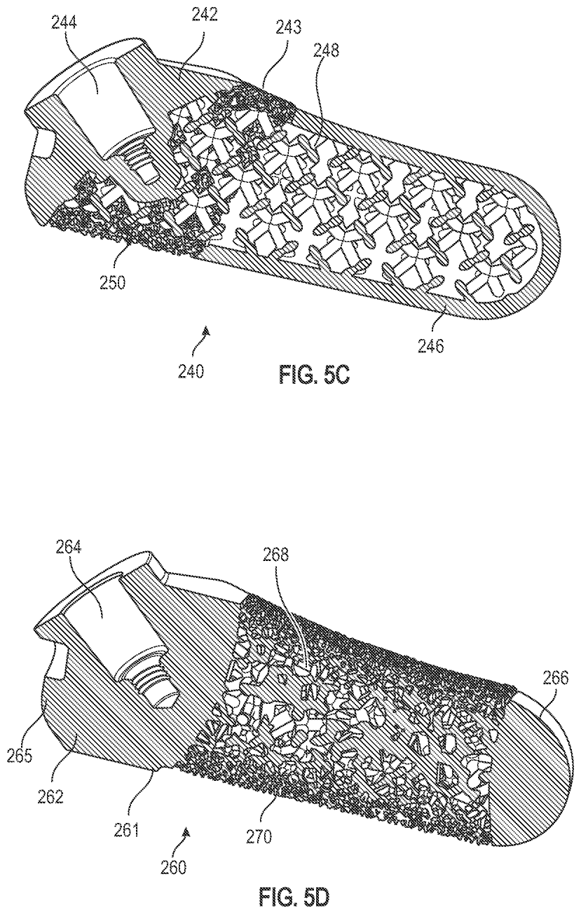

FIGS. 5A, 5B, 5C, and 5D illustrate cross-sections of example stems 200, 220, 240), and 260, respectively, in accordance with at least one embodiment of this disclosure. FIG. 6 illustrates a lattice structure 300, in accordance with at least one embodiment of this disclosure. FIGS. 5A-6 are discussed below concurrently. The stem 200, the stem 220, the stem 240), and the stem 260 can each be representative of, but not limited to, the stem 108 or the stem 130 shown in, and discussed with respect to, FIG. 1 or FIG. 2, respectively. With respect to FIG. 5A, the stem 200 (FIG. 5A) can include an exterior portion 202 (FIG. 5A). The exterior portion 202 can generally an outer portion or section of the stem 200. For example, the exterior portion 202, in some examples, can be generally representative of the exterior portion 142 (FIGS. 3B-4D).

The exterior portion 202 can define an outer surface 203 (FIG. 5A). The outer surface 403 can be an outermost surface of the stem 200. In FIG. 5A, the exterior portion 202 can be of solid construction, and the outer surface 203 can be a solid or otherwise continuous non-porous surface. The exterior portion 202 can define a cavity 204 (FIG. 5A). The cavity 204 can be a non-solid or otherwise non-continuous interior portion or space located within the stem 200. In some examples, such as shown in FIG. 5A, the stem 200 can include a lattice structure 206 (FIG. 5A) defined or formed within the cavity 204. The lattice structure 206 can be generally representative of a variety of non-solid or porous three-dimensional structures adapted to be lighter, and more flexible, than solid portions or segments of the stem 200, such as, but not limited to, the exterior portion 202.

For example, the lattice structure 206 can include, or otherwise be formed by, a plurality of solid portions 211 (FIG. 5A) separated from one another by a plurality of void spaces 212 (FIG. 5A). In various examples, each the plurality of solid portions 211 can form various three-dimensional shapes, such as, but not limited to, cylindrical, triangular, rectangular, pentagonal, or hexagonal cross-sectional shapes. Additionally, each the plurality of solid portions 211 can be connected to one another at various angles or intervals to form, for example, but not limited to, repeating patterns, such as one or more tessellations within the lattice structure 206, or one or non-repeating patterns within the lattice structure 206.

In some examples, the lattice structure 206 can include, or can be divided into, a plurality of segments 210 (FIG. 5A). Each of the plurality of segments 210 can generally be defined as a cross-sectional portion or area of the lattice structure 206 each having different parameters or arrangements relative to one another. For example, a size and shape of one or more of, or a pattern formed by, the plurality of solid portions 211 located within a first segment 214 of the plurality of segments 210 and a size and shape of one or more of, or a pattern formed by, the plurality of solid portions 211 located within a second segment 216 of the plurality of segments 210. The plurality of segments 210 can include various numbers of individual segments, such as, but not limited to, two, three, four, five, six, seven, or eight individual segments. In one such example, as shown in FIG. 3, a lattice structure 300 (FIG. 6), which can be representative of the lattice structure 206 of the stem 200, or any other lattice structures described or shown in the present disclosure, can include a first segment 302 (FIG. 6) and a second segment 304 (FIG. 6).

The first segment 302 can define a first diameter D1 (FIG. 6) and a first length L1 (FIG. 6) and the second segment 304 can define a second diameter D2 (FIG. 6) and a second length L2 (FIG. 6) which can be similar or different to the first length L1. The first diameter D1 can be greater than or less than the second diameter D2, and the progression or difference in diameter therebetween, or between other diameters of other segments of a plurality of segments of a lattice structure in accordance with the present disclosure, can be a linear progression, a polynomial progression, a logarithmic progression, or can otherwise be represented by other mathematical sequences or patterns. In the above examples, various parameters of the lattice structure 206, such as including, but not limited to, the size and shape of one or more of, or a pattern formed by, the plurality of solid portions 211 located within one or more segments of the plurality of segments 210, the number of individual segments of the plurality of segments 210, or the diameter and length of each segment of the plurality of segments 210 can be optimized, or otherwise determined, for an individual patient or a plurality of patients during the design process of the present disclosure, which is discussed in detail below with respect to FIGS. 8-11.

The exterior portion 202 of the stem 200 can also include a head section 209 (FIG. 5A) defining a socket 208 (FIG. 5A). The head section 209 can generally be a portion of the stem 200 adapted to receive, for example, a threaded or tapered portion of the articulation component 110 (FIG. 1) or the articulation component 132 (FIG. 2) to facilitate fixation therebetween. The head section 209 can generally have a solid or non-porous construction proximate to the socket 208, such as to resist deformation and provide support for the articulation component 110, the articulation component 132, or a variety of other prosthetic components adapted to engage with the socket 208 such as, but not limited to, a prosthetic femoral head or cup.

With respect to FIG. 5B, the stem 220 can include an exterior portion 221 (FIG. 5B). The exterior portion 221 can be similar to the exterior portion 202 of the stem 200 shown in FIG. 5A, at least in that the exterior portion 221 can be of solid construction and can define an outer surface 223 (FIG. 5B). During the design process of the present disclosure, discussed in detail with respect to FIGS. 8-11 below, a ratio of porous or lattice construction to solid construction can be optimized or otherwise determined for an individual patient, or can be varied between patients, to provide for a non-solid metal construction in regions of lower stress within an implanted bone and a solid metal construction in regions of higher stress.

In one such example, the stem 220 can include a head section 222 (FIG. 5B) that is solid around a greater cross-sectional area or portion proximate of a socket 224 (FIG. 5B), relative to the head section 209 (FIG. 5A) of the stem 200 (FIG. 5A), such as to more effectively resist deformation and provide additional support for the articulation component 110 (FIG. 1), the articulation component 132 (FIG. 2), or a variety of other prosthetic components, supported by the socket 208 (FIG. 5A) of the stem 200. In such an example, it is to be appreciated that a cavity 228 (FIG. 5B) enclosing a lattice structure 226 (FIG. 5B) of the stem 220 can be smaller than the cavity 204 (FIG. 5A) of the stem 200.

In other examples, various or other parts or sections of the stem 220, or other stems of other implants, can be reinforced or otherwise constructed differently relative to the stem 200 to optimize the flexibility or stiffness of such stems for an individual patient, such as by, but not limited to, by defining a different thickness or distance, such as measured between the outer surface 223 and a cavity 228 (FIG. 5B) defined by the exterior portion 221, than a thickness or distance measured between the outer surface 203 and the cavity 204 of the exterior portion 202. In such examples, it is to be appreciated that the size and shape of any of a plurality of segments 230 (FIG. 5B) of a lattice structure 226 (FIG. 5B) formed or enclosed within the cavity 228 can be varied in proportion to the size and shape of the cavity 228, such as by having different diameters, different lengths, or different orientations relative to the plurality of segments 210 (FIG. 5A) of the lattice structure 206 (FIG. 5A).

With respect to FIG. 2C, the stem 240) (FIG. 2C) can include an exterior portion 246 (FIG. 5C) including a head section 244 defining a socket 244; and, the head section 244 can be different to the exterior portion 202 and the head section 209 of the exterior portion 202 shown in FIG. 5A, or the exterior portion 221 or the head section 222 of the stem 220) shown in FIG. 5B, at least in that the exterior portion 246 can be at least partially made from a non-solid or porous construction. During the design process of the present disclosure, discussed in detail below with respect to FIGS. 8-11, a ratio of non-solid or porous construction to solid construction of an implant can further be optimized, such as by connecting the head section 242 to the exterior portion 246 with a lattice structure 248 and/or a porous metal section 250. In one such example, some portions or segments of an outer surface 243 (FIG. 2C) of the exterior portion 246 can be defined by the porous metal section 250) (FIG. 2C).

The porous metal section 250 can generally be a textured or a patterned three-dimensional structure adapted to help facilitate post-operative bone ingrowth and vascularization. The porous metal section 250 can form, or be integrated with, portions, areas, or one or more segments of the lattice structure 248 and/or can be deposited onto the lattice structure 248. In some examples, the porous metal section 250) can be realized using Zimmer Biomet OsseoTi® Porous Metal Technology, which uses human CT imaging data in combination with 3D printing technology to build a structure that mimics the architecture of human cancellous bone. In other examples, the porous metal section 250) can be realized using Zimmer Biomet Proximal PPS® Porous Plasma Spray.

With respect to FIG. 2D, the stem 260 (FIG. 5D) can include an exterior portion 261 (FIG. 5D) including a proximal portion 265 (FIG. 5D) and a distal portion 266 (FIG. 5D). The proximal portion 265 can include a head section 262 (FIG. 5D) defining a socket 264 (FIG. 5D). During the design process of the present disclosure, discussed in detail below with respect to FIGS. 8-11, a ratio of non-solid or porous construction to solid construction of an implant can additionally be determined for an individual patient. For example, the proximal portion 265 and the head section 262 can be connected to the distal portion 266 by a lattice structure 268 (FIG. 5D). The lattice structure 268 can be different to the lattice structure 206, the lattice structure 226, or the lattice structure 248, in that the lattice structure 268 can be at least partially constructed from, or formed by, a porous metal section 270) (FIG. 5D).

In some examples, the porous metal section 250 can be realized using Zimmer Biomet OsseoTi® Porous Metal Technology, which uses human CT imaging data in combination with 3D printing technology to build a structure that mimics the architecture of human cancellous bone. In other examples, the porous metal section 250 can be realized using Zimmer Biomet Proximal PPS® Porous Plasma Spray. Finally, in some examples, the density of the porous metal section 250 (FIG. 2C) and the porous metal section 270 (FIG. 2D), and/or a ratio of the porous metal section 270 to solid portions of the lattice structure 248 (FIG. 2C) or the lattice structure 268 (FIG. 2D) can also be determined to further optimize flexibility or stiffness of such stems for an individual patient for an individual patient. In this way, the stems 200, 220, 240, 260, or other stems including lattice structures or porous metal sections in accordance with the present disclosure, can significantly reduce the stress-shielding caused by entirely or completely solid implants, and in, turn, reduce the likelihood of bone resorption and peri-prosthetic fractures associated therewith.

In view of all the above, during the design process of the present disclosure, discussed in detail with respect to FIGS. 8-11 below, any of a wide range of various implant parameters of the stems 200, 220, 240, and 260, such as including, but not limited to, any physical characteristics of exterior portions, outer surfaces, cavities, lattice structures, porous metal sections or porous surfaces, sockets, head sections, or other features, can be optimized. In any of the above examples, the stems 200, 220, 240, and 260, or the stems of other implants, can be made from various materials, such as, but not limited to, ceramics, metals, such as stainless steel, titanium, or metal alloys including cobalt or chromium, through a variety of manufacturing techniques, such as, but not limited to, three-dimensional printing, metallic molding, machining, or additive manufacturing.

FIG. 7A illustrates a cross-section of an example baseplate 400, in accordance with at least one embodiment of this disclosure. FIG. 7B illustrates a cross-section of an example baseplate 420, in accordance with at least one embodiment of this disclosure. FIGS. 7A-7B are discussed below concurrently. The baseplate 400 and the baseplate 420 can each be representative of, but not limited to, the baseplate 134 shown in, and discussed with respect to, FIGS. 2 & 3-4D. With respect to FIG. 7A, the baseplate 400 (FIG. 7A) can include an exterior portion 402 (FIG. 7A). The exterior portion 402 can generally an outer portion or section of the baseplate 400. The exterior portion 402 can define an outer surface 403. The outer surface 403 can be an outermost surface of the baseplate 400.

In some examples, such as shown in FIG. 7A, the exterior portion 402 can be of solid construction, and the outer surface 403 can be a solid or otherwise continuous non-porous surface. The outer surface 403 can also include a solid contacting portion 405. The solid contacting portion 405 can be a surface area of the outer surface 403 sized and shaped to contact and engage a scapular surface, such as, but not limited to, the glenoid surface 146 (FIGS. 3A-3B). In some examples, the solid contacting portion 405 can be representative of the outer contacting surface 144 (FIGS. 4B-4D) of the exterior portion 142 (FIGS. 3B-4D) of the baseplate 134. In some such examples, the contacting portion 405 can include, or be coated with, a porous surface 407 (FIG. 7A). The porous surface 407 can be, in some examples, realized using Zimmer Biomet OsseoTi® Porous Metal Technology, which uses human CT imaging data in combination with 3D printing technology to build a structure that mimics the architecture of human cancellous bone. In other examples, the porous surface 407 be realized using Zimmer Biomet Proximal PPS® Porous Plasma Spray.

The exterior portion 402 can define a cavity 404 (FIG. 7A). The cavity 404 can be a non-solid or otherwise non-continuous interior portion or space located within the baseplate 400. In some examples, the baseplate 400 can include a lattice structure 406 (FIG. 7A) defined or formed within the cavity 404. The lattice structure 406 can be generally representative of a variety of non-solid or porous three-dimensional structures adapted to be lighter, and more flexible, than solid portions or segments of the baseplate 400, such as, but not limited to, the exterior portion 402. For example, the lattice structure 406 can include, or otherwise be formed by, a plurality of solid portions 411 (FIG. 7A) separated from one another by a plurality of void spaces 412 (FIG. 7A). In various examples, each the plurality of solid portions 411 can form various three-dimensional shapes, such as, but not limited to, cylindrical, triangular, rectangular, pentagonal, or hexagonal cross-sectional shapes. Additionally, each the plurality of solid portions 411 can be connected to one another at various angles or intervals to form, for example, but not limited to, repeating patterns, such as one or more tessellations within the lattice structure 406, or one or non-repeating patterns within the lattice structure 406.

In some examples, the lattice structure 406 can include, or can be divided into, a plurality of segments 410 (FIG. 7A). Each of the plurality of segments 410 can generally be defined as a cross-sectional portion or area of the lattice structure 406 each having different parameters or arrangements relative to one another. For example, a size and shape of one or more of, or a pattern formed by, the plurality of solid portions 411 located within a first segment 414 of the plurality of segments 410 and a size and shape of one or more of, or a pattern formed by, the plurality of solid portions 411 located within a second segment 416 of the plurality of segments 410.

In one such example, such as shown in FIG. 3, a lattice structure 300 (FIG. 6), which can be representative of the lattice structure 406 of the baseplate 400, or any other lattice structures in accordance with the present disclosure, can include a first segment 302 (FIG. 6) and a second segment 304 (FIG. 6). The first segment 302 can define a first diameter D1 (FIG. 6) and a first length L1 (FIG. 6) and the second segment 304 can define a second diameter D2 (FIG. 6) and a second length L2 (FIG. 6) which can be similar or different to the first length L1. In other examples, the plurality of segments 410 can include other numbers of segments, such as, but not limited to, three, four, five, six, seven, or eight individual segments.

The exterior portion 402 of the baseplate 400 can define a socket 408 (FIG. 7A). The socket 408 can be adapted to receive, for example, a threaded or tapered portion of the glenosphere 136 (FIG. 2), or other portions or features of various articulation components, to facilitate a threaded or press-fit fixation therebetween. In some examples, the socket 408 can be representative of the socket 150 (FIGS. 3B & 4A) of the baseplate 134 (FIGS. 3B-4B). The exterior portion 402 can generally have a solid or non-porous construction proximate to the socket 408, such as to resist deformation and provide support for the glenosphere 136 or a variety of other prosthetic components adapted to engage with the socket 208 such as, but not limited to, a prosthetic femoral head or cup. Such a solid or non-porous construction can be varied during the design process of the present disclosure to be optimized for an individual patient. For example, the cross-sectional area of the solid or non-porous construction of the exterior portion 402 proximate to the socket 408, can be reduced, such as illustrated by the head section 209 (FIG. 5A), or increased, such as illustrated by the head section 222 (FIG. 5B). In such examples, it is to be appreciated that the cavity 404 enclosing the lattice structure 406 can shrink, or decrease in size or volume, as the cross-sectional area of the solid or non-porous construction of the exterior portion 402 increases.

With respect to FIG. 7B, the baseplate 420 can include an exterior portion 421 (FIG. 7B). The exterior portion 421 can be similar to the exterior portion 402 of the baseplate 400 shown in FIG. 7A, at least in that the exterior portion 421 can be generally representative of the exterior portion 142 (FIGS. 3B-4D) and can define an outer surface 422, a cavity 424. a lattice structure 406, and a socket 428 adapted to receive, for example, a threaded or tapered portion of the glenosphere 136 (FIG. 2), or other portions of various articulation components, to facilitate a threaded or press-fit fixation therebetween. During the design process of the present disclosure, discussed in detail with respect to FIGS. 8-11 below, a ratio of porous metal construction to solid metal construction can be determined to help optimize the flexibility or stiffness of an implant for an individual patient, such as in addition to determining the cross-sectional area of the solid construction of the exterior portion 402 proximate to the socket 428, to provide for a non-solid metal construction in regions of lower stress within an implanted bone and a solid metal construction in regions of higher stress by constructing or reinforcing various parts or sections of the baseplate 400 (FIG. 7A) or the baseplate 420 differently between various patients.

First, for example, such as in contrast to the baseplate 400 (FIG. 7A), the baseplate 420 can include a porous metal section 430. The porous metal section 430) can extend outwardly from the lattice structure 426 to form a porous contacting portion 425 of the outer surface 422, such as alternatively to the solid contacting portion 405 (FIG. 7A). In some examples, the porous contacting portion 425 can be representative of the outer contacting surface 144 (FIGS. 4B-4D) of the exterior portion 142 (FIGS. 3B-4D) of the baseplate 134. In another example, the lattice structure 426 of the baseplate 420 can, in contrast to the lattice structure 406 (FIG. 7A), be at least partially constructed from, or otherwise formed by, the porous metal section 430. Second, the cross-sectional area of the cavity 424 or the lattice structure 426, relative to the cross-sectional area of the cavity 404 or the lattice structure 406, can be optimized for an individual patient. Third, in some examples, the density of the porous metal section 430 or the lattice 426 and/or a ratio of the porous metal section 430 to solid portion, such as a plurality of solid portions 411 (FIG. 5A) of the lattice structure 426 can be optimized for an individual patient. In this way, the baseplates 400 and 420, or other baseplates including lattice structures or porous metal sections in accordance with the present disclosure, can significantly reduce the stress-shielding caused by entirely or completely solid implants, and in, turn, reduce the likelihood of bone resorption and peri-prosthetic fractures associated therewith.

In view of all the above, during the design process of the present disclosure, discussed in detail with respect to FIGS. 8-11 below; any of a wide range of implant parameters of the baseplates 400 and 420, or other baseplates of other glenoid implants, such as including, but not limited to, any physical characteristics of exterior portions, outer surfaces, cavities, lattice structures, porous metal sections or porous surfaces, sockets, head sections, or other features, can be optimized. In any of the above examples, the baseplates 400 and 420 can be made from various materials, such as, but not limited to, ceramics, metals, such as stainless steel, titanium, or metal alloys including cobalt or chromium, through a variety of manufacturing techniques, such as, but not limited to, three-dimensional printing, metallic molding, machining, or additive manufacturing.

FIG. 8 illustrates a method 500 for manufacturing an implant, in accordance with at least one example of this disclosure. The method 500 can, in some examples, be generally representative of the design process as discussed with reference to any of FIGS. 1-7B above. The steps or operations of the method 500 are illustrated in a particular order for convenience and clarity; many of the discussed operations can be performed by multiple different actors, devices, or systems. It is understood that subsets of the operations discussed in the method 500 can be attributable to a single actor, device, or system and can be considered a separate standalone process or method.

The method 500 can include a first stage 502. The first stage 502 can include receiving patient-specific data related to a bone. Receiving the patient-specific data can include receiving, at a computing device, one or more scans, such as including, but not limited to, X-rays, CT scans, fluoroscopic images, or other imaging data of a patient's anatomy relating to one or more bones of the patient, such as, a humerus or a scapula of a shoulder joint of the patient, a femur or pelvis of a hip joint of the patient, or bones associated with various other joints of the patient.

The first stage 502 can also include extracting design constraints for an implant for the patient, such as, but not limited to, a stem or a baseplate of a shoulder or hip implant. Next, using various image analysis techniques, physical characteristics or features of the patient's anatomy, such as, but not limited to, the bone density (based on pixel values that may represent bone density) of one or more bones of the patient, various geometric parameters of one or more bones of the patient (e.g., the dimensions of features or surfaces thereof), or the relative anatomical locations of bone, ligament, or tendons within the patient, can be determined or otherwise extracted. The bone density values, the relative anatomical locations, or the geometric parameters can be used as design constraints (e.g., implant parameters) for the implant for the patient, as they can be used to represent, for example, but not limited to, maximum geometric dimensions or constraints including a curvature. diameter, length, or otherwise a size and shape, for various surfaces or other features of the implant for the patient.

The method 500 can include a second stage 504. The second stage 504 can include receiving implant data related to an implant. Receiving the implant data can include determining maximum, minimum, and nominal values for design constraints (e.g., implant parameters) determined or otherwise extracted from the patient-specific imaging data related to the patient's anatomy during the first stage 502. For example, based on any of the bone density values, relative anatomical locations, or geometric parameters, determined or extracted from the patient-specific imaging data during the first stage 502, maximum, minimum, and nominal values for various geometrical dimensions, such as including, a curvature, diameter, length, or otherwise an overall size and shape, for various features or surfaces of the implant for the patient, can be determined.

The nominal values can represent customary, or otherwise previously used, values that have been suitable or adequate for other patients having, or otherwise exhibiting, similar physical characteristics extracted or determined from imaging data. For example, if one or more previous patients exhibiting similar patient-specific imaging data were normally, or have otherwise been, fitted with an implant having X. Y, and Z implant parameters in the past, then the nominal values for the individual patient of the method 500 can be X. Y, and Z. In some examples, the second stage 504 can include receiving implant data from a database or repository for storing parameters of various stock or otherwise standardized implants routinely manufactured by an implant manufacturer. In such examples, the stock or standardized implants can represent the nominal values for various implant designs. In some examples, the second stage 504 can further include entering data, such as entered by a surgeon or other medical staff, for the implant for the patient based on a review of the patient-specific imaging data obtained during the first stage 502 and personal experience treating patients having similar imaging or scan data.

In some examples, the second stage 504 can include receiving implant parameters for a lattice structure and/or one or more porous metal sections of the implant for the patient. For example, when the individual patient of the method 500 has physical characteristics such as, but not limited to, an age, one or more lifestyle factors (such as, but not limited to, does the patient play sports, does the patient carry heavy weights frequently, etc.), one or more bones density values, relative anatomical locations, geometric parameters, or other attributes, the size, shape, and relative location of each of a plurality of segments of the lattice structure of the implant can be determined or estimated to provide and optimized combination of rigidity and flexibility for the implant, to thereby minimize the likelihood of bone resorption and peri-prosthetic bone fractures while also promoting post-operative healing.

The method 500 can include a third stage 506. The third stage 506 can include determining an optimal implant design using the patient-specific data related to the bone and the implant data related to the implant. Determining an optimal implant design can include a first step 508 and a second step 510. The first step 508 can include generating one or more designs for the implant for the patient, such as, but not limited to, one or more design options for a stem or a baseplate of the implant. Generating the plurality of designs can include passing, as inputs, various patient related parameters, such as determined or extracted from the patient-specific imaging data during the first stage 502, such as, among others, bones density values, relative anatomical locations, geometric parameters, an age, or quantified lifestyle factors, into a machine learning (hereinafter “ML”) model (e.g., a machine learning artificial intelligence model), and receiving or otherwise obtaining implant parameters as outputs from the ML model. In some examples, the generating the one or more designs can include generating a plurality of different design options by generating a number of different implant designs, using the ML model, that have different permutations of various implant parameters.

In some examples, the first step 508 can include selecting one or more standardized or standard line implant designs, such as, but not limited to, one or more standardized shaped and sized stems or one or more standard sized and shaped baseplates for the implant, from a catalog or repository of designs based on the patient specific data and the implant data.

The second step 510 can include evaluating the designs for the implant for the patient (e.g., the one or more design options or the plurality of design options). Evaluating the designs can include performing finite element analysis (hereinafter “FEA”), and/or Musculoskeletal modeling, on each of the one or more design options for the implant. For example, during FEA or Musculoskeletal modeling, a stem or baseplate of the implant, or various other portions of the implant, can be divided into a plurality of segments, and one or more meshes generated along with boundary conditions can be applied thereto. Further, during the FEA or Musculoskeletal modeling, a fit condition between the bone of the patient (e.g., the bone to receive or engage with the implant) can be simulated. For example, based on, among others, bone quality or health of the bone, a reamer size, the size and shape of the implant, including various surfaces or features thereof, the estimated fit of the implant in the bone after reamed or shaping of the bone can be simulated. This can determine, among others, if the implant is too big or small, or is otherwise shaped correctly to engage with, the reamed bone. For instance, for relatively soft bone tissue, a reamer may remove too much bone tissue for a given stem or baseplate size or shape; and, by virtue of simulating the reaming or shaping process, a likely size of a hole or cavity created by reaming or a likely the depth of reaming over a particular time or with a particular force, can be estimated and compared to the size and shape of various surfaces or features of the implant.

Subsequently, in some examples, implant data representing any of various portions, segments, or areas of the implant can be changed from a first value to a second value, and the fit condition between the bone and the implant can be re-simulated, such as until a quality or suitable estimated fit is achieved. One example of an implant parameter that can be varied or changed from a first value to a second value can include, but is not limited to, a value that represents a solid metal material or construction, to be changed to a value that represents a lattice or a porous metal material or construction. For example, among others, portions of a stem or a baseplate of the implant can be conventionally, or in stock form, made of solid metal construction, and as such, do not flex or otherwise flex minimally. In such an example, and one or more first values that represent such solid portions can be changed to one or more second values indicating that such solid portions are now, or will be, porous portions (e.g., a lattice structure or a porous metal section as discussed with reference to at least FIGS. 5A-7B) that are more flexible or less dense than the previously solid portions.

In such examples, the more flexible nature of the porous portions can allow for a better fit of the implant by bringing stress forces or stress distributions within the implanted bone of the patient closer to those experienced in a natural or non-implanted bone, such as by allowing portions of the implant to flex slightly to account for differences in bone density, surface irregularities, or other conditions of, or within, the bone of the patient that may be present before a surgical procedure or that can created by reaming or bone shaping. In this way, stress-shielding within the bone of the patient can be reduced, and in, turn, the likelihood of bone resorption and peri-prosthetic fractures associated therewith can be decreased.

In further examples, the ratio of a lattice structure relative to a porous metal section of an implant can also be determined or changed by performing simulations using the ML model. For example, one or more first values that represent solid portions within a segment of a plurality of segments of a lattice structure can be changed to one or more second values indicating that such solid portions within the segment are now, or will be, made from porous metal or can otherwise be part of the porous metal section, such as to help improve bone ingrowth into the lattice structure or reduce the density or stiffness of the lattice structure. Still further examples of implant parameters that can be changed from a first value to a second value, but are not limited to, one or more dimensions for a portion of a stem or a baseplate of the implant, such as for example, a degree of taper of a stem, a size and shape of an exterior, exterior portion, outer surface, or contacting surface of a stem or baseplate. In such an example, such implant parameters can be changed from a first value to a second value, and the revised fit condition between the bone and stem can be simulated using the ML model to determine if an improved implant fit has been obtained.

In some examples, the second step 510 can also include determining regions of the bone of the patient with optimal stresses based on simulated loading of the implant. For example, during FEA or Musculoskeletal modeling, various loads and/or moments may be placed on or applied to an implant design. In an example, loading is simulated with the implant virtually implanted in the target bone. Based on the loading and moments, stresses within the bone can be determined. Areas where stresses are above a predetermined level (bone stresses in an intact bone) can be identified, and implant parameters of a stem or a baseplate of the implant for the patient can be changed to lower or minimize the stresses. Alternatively, areas where stresses are below a predetermined level (bone stresses in an intact bone) can be identified, and implant parameters of a stem or baseplate of the implant for the patient can be changed to increase the stresses. For instance, in an area of the implant having a solid metal construction, a stress concentration can form based on loading of the stem. The area of the implant can then be changed from a solid metal construction to a porous metal construction (e.g., a lattice structure and/or a porous metal section), to reduce or eliminate the stress concentration by allowing for flexing in, or deflection of, a stem, baseplate, or other portions or features of the implant.

In some examples, the second step 510 can include optimizing a lattice structure of the implant in portions or segments of the implant with minimal stresses. For example, portions of the implant for the patient that are made of, or otherwise include, lattice structures or porous metal sections can have different parameters which can be optimized by loading of, or applying forces or moments to, an implant design. For instance, as part of FEA or Musculoskeletal modeling, the number of segments of a plurality of segments the lattice structure includes, the diameter, length, and shape of each of a plurality of solid portions of the lattice structure, the amount of the lattice structure formed from a porous metal material or section, or the density of pour metal material or section, can be altered from one iteration to the next to determine stress concentrations within the bone of the patient. Based on these stress concentrations, various implant parameters that result in minimized or reduced stresses or otherwise reduce or minimize such stress concentrations can be selected.

In some examples, the second step 510 can also include determining a size, location, number of, or other attributes of one or more placement fixtures of the implant for the patient. For example, if portions of the implant are made of or include a porous metal construction (e.g., a lattice structure or porous metal section), or if one or more surfaces of the implant include a porous surface, such as a surface having a porous or material or layer deposited thereon, to help promote bone ingrowth, the location and coverage area of the porous coating, and thereby the size of the one or more placement fixtures, can be simulated and optimized using the ML model. Further, various attributes or characteristics of fixation devices or features of the implant, such as, among others, head sections including a socket, fins or pegs, or suture anchor holes or a plurality of bores for receiving bones screws or other fixation devices, can be altered from one implant design to another, and the reactions to various applied loadings and moments can then be simulated using the ML model to minimize stresses generated by, or concentrated near, such fixation devices or features.

The method 500 can include a fourth stage 512. The fourth stage 512 can include determining if the implant design is optimal for an individual patient. In some examples, an implant design can be considered optimal when stresses generated within the bone are optimized to minimize stress concentrations therein. For example, if a first implant design results in average stresses within the bone being X and a second implant design results in average stresses within the bone being Y, with Y being greater than X, then Y can be said to be a less optimal design than X. If a third implant design results in average stresses that are lower than X, the Z can be said to be a more optimal design to X. The comparison of stresses can be repeated for each of the designs generated in an iterative process until an optimal implant design is found. The fourth stage 512 can also include selecting an implant design, such as indicated by reference number 514. For example, once an optimal implant design is found, the optimal implant design can be selected for manufacturing.

The method 500 can include the fifth stage 516. The fifth stage 516 can include constructing the implant for the patient. Constructing the implant for the patient can include exporting the optimal design parameters for a selected optimal implant, such as determined during the third stage 506 and the fourth stage 512. For example, the optimal design parameters of the optimal implant design can be exported or otherwise transmitted via computing device to an automated manufacturing device such as, but not limited to, a three-dimensional printer, a computer numerical controlled (CNC) milling machine, or other devices or systems. In some examples, the implant for the patient can be constructed using additive manufacturing, such as built layer by layer from titanium or cobalt-chrome. In some examples, the fifth stage 516 can include post-processing, such as surface finishing or sterilization, to help ensure that the implant meets specifications and is safe for implantation, which may include fatigue testing or material characterization. In view of all the above, the method 500 can represent a design process capable of significantly reducing the stress-shielding caused by solid implants, and in, turn, reduce the likelihood of bone resorption and peri-prosthetic fractures associated therewith.

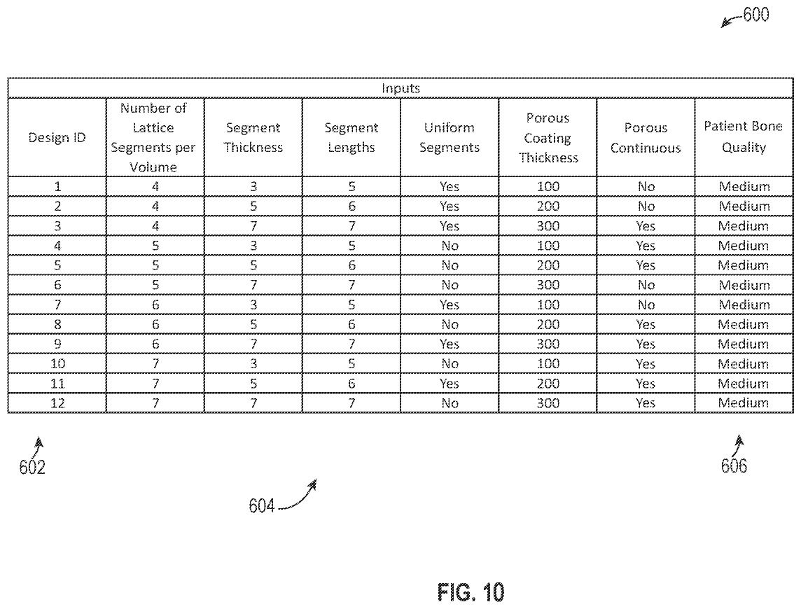

FIG. 10 shows a table 600 having example inputs that can be used as design parameters. For example, and as shown in FIG. 10, each implant design can be assigned a design ID 602. Each implant design can have various properties that can be inputs 604 to a model as disclosed herein. Inputs 604 can include parameters for lattice specifications and locations and parameters for fixation components as well as parameters related to a patient, such as bone quality. For example, inputs 604 can include parameters related to bone quality as indicated by reference numeral 606. As disclosed herein, the inputs 604 can be numerical values, such as dimensions related to lattice design and placement as well as porous coating design, etc. Inputs 604 can also be string values. For example, the bone quality of the bone of the patient can be represented or quantified as strings as shown in FIG. 10. The bone quality can also be represented or quantified by various numeric values. For instance, among other intervals, low bone quality can be represented as 1, medium bone quality can be represented as 2, and high bone quality can be represented as 3. Once the various implant designs are generated during the first step 508 of the third stage 506, each of the implant designs can be evaluated numerically, such as by using FEA or Musculoskeletal modeling during the second step 510 of the third stage 506 to provide outputs.

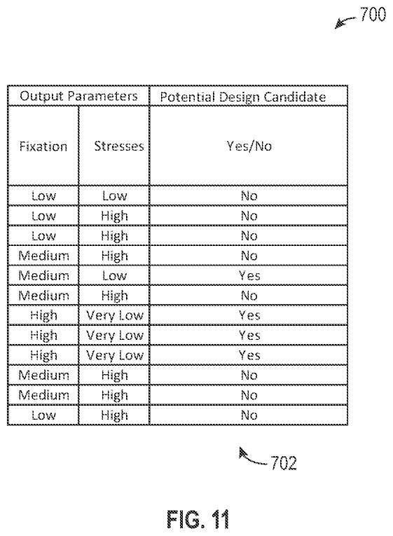

FIG. 11 shows a table 700 with example outputs 702. The outputs 702 can be presented as strings or numerical values. For example, as shown in FIG. 11, the outputs 702 can be presented as a table (or array) of strings for a surgeon to review. The outputs 702 can be derived and/or represented by numerical values. For example, among other intervals, a very high fixation strength or a very high quality fit between the implant and the bone of the patient can be represented as 1, high fixation or a high quality fit between the implant and the bone of the patient can be represented as 2, a medium quality fit between the implant and the bone of the patient can be represented as a 3, and a low quality fit between the implant and the bone patient can be represented as 4. The fixation strength or fit quality can be based on movement of one or more fixation components, such as, but not limited to, a plurality of bone screws, or a press or cement fit between the implant and the bone, as determined during FEA. Stresses can also be determined during the FEA or Musculoskeletal modeling process. These values may be numerical and converted to strings for presentation to a surgeon to review. While tables 600 and 700 show only twelve design options, it is to be appreciated that the data set can include thousands, tens of thousands, or hundreds of thousands of implant design options. Thus, the evaluation of designs during the second step 510 (FIG. 8) of the third stage 506 (FIG. 8) of the method 500 (FIG. 8) can include evaluating multiple implant designs and selecting a preset number of potentially optimal implant designs for presentation to the surgeon, one of which can be selected by the surgeon during the fourth stage 512 (FIG. 8) as shown by reference number 514 (FIG. 8).

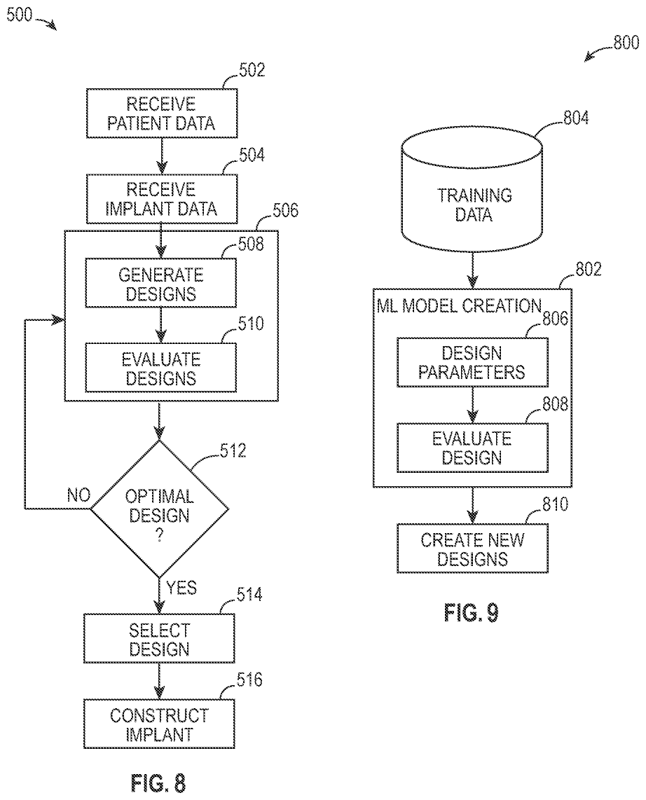

FIG. 9 illustrates an example method 800 for designing prosthetic implant designs, in accordance with at least one example of this disclosure. The method 800 can, in some examples, be generally representative of the design process (e.g., the method 500), as discussed with reference to any of FIGS. 1-7B above. The steps or operations of the method 800 are illustrated in a particular order for convenience and clarity; many of the discussed operations can be performed by multiple different actors, devices, or systems. It is understood that subsets of the operations discussed in the method 800 can be attributable to a single actor, device, or system and can be considered a separate standalone process or method.

The method 800 can include a first stage 802 and a second stage 810. The first stage 802 can include creation of an ML model (e.g., a machine leaning artificial intelligence model). In some examples, the first stage 802 can include a first step 806 and a second step 808. In some examples, the first step 806 can include receiving or otherwise using inputs from a training data set 804. In some examples, the first step 806 can include defining implant design parameters and the second step 808 can include evaluating one or more implant designs. The first step 806 and the second step 808 can be similar to, or can otherwise include, any of various aspects of, the first stage 502, the second stage 504, and the third stage 506, respectively, discussed with respect to FIG. 8 above. In other words, the ML model creation operation 802 can utilize training data that can include any of the parameters discussed above in reference to FIG. 8 related either patient data or implant data or data derivable from the patient data, implant data or a combination thereof.

The implant design parameters defined or otherwise determined during the first step 806 can be affected by, or can be based on, the type or style of surgical procedure to be undertaken by the individual patent. For example, during the first stage 802, receiving or using inputs from the training data set 804 or other input data, can include input data related to or identifying the surgical procedure. As such, creation of the ML model can include defining or inputting the surgical procedure. Additionally, for example, depending on how a bone of the patient in which the implant is to be received in or affixed to, is to be cut, resected, or otherwise shaped, more or less of the bone of the patient, such as a portion of the bone remaining after resection or surface shaping, can be included in the implant design parameters input into the ML model during its creation. Also, the anticipated placement or positioning of the implant on, or within, the bone of the patient can dictate how much space may be available for movement or placement of the implant in various directions within, or on, the bone, during a surgical procedure.

The first stage 802 can include generating a model. The model can allow the various inputs (e.g., the implant parameters), such as, but not limited to, the inputs shown in table 600 (FIG. 10) to be entered thereinto. The model can be used to output a number, this example, that corresponds to one of the various implant designs generated, such as during the third stage 506 (FIG. 8). For example, the various inputs can be entered into the ML model, and the ML model can, in return, output a number. X. The number X can correspond to a model number of a stock or standardized implant. To select the stock implant, for example, one or more lookup tables can be used to select the implant from a database or repository. The various inputs can be part of a query to a database or repository. Using the various inputs, the ML model can return or output the implant design that had the closest inputs to the various inputs when a stock or standardized implant design was created or designed.

For example, each of the stock or standardized implants can have preset specifications that were used as part of its creation or generation. These specifications can be stored in the database or repository as query variables for selecting a stock or standardized implant. In some examples, the database or repository can be a K-nearest-neighbor (KNN) database. As such, a Euclidean distance between the various inputs for a specific patient and specifications for each of the stock or standardized designs can be calculated, and a specific or individual design with the minimum distance can be returned as the optimal implant design for the patient to be selected and manufactured during the fourth stage 512 (FIG. 8) and the fifth stage 516 (FIG. 8).

In some examples, the first stage 802 can include generating an initial guess for the implant parameters for the ML model based on the various inputs. The parameters of the ML model can be optimized from the initial guess for the ML model and the plurality of input parameters. For example, for a given bone size, or a given bone quality, of the bone of the patient, an initial guess for a number, location, or other attributes of various fixation devices or features, such as, among others, head sections including a socket, a number of fins or pegs, fin thickness, fin height, a number of holes in the fins, suture anchor holes or a plurality of bores for receiving bones screws, or a wide variety of other parameters, can be made, and implant designs can be created based thereon. These implant designs, which can be solid models, can then be subjected to FEA or Musculoskeletal modeling to confirm, for example, a quality fit or suitable fixation strength between the implant and the bone of the patient, a ratio of porous metal construction to solid metal construction within the implant, or other optimized parameters of the implant for the patient.

The training data 804 can be divided into various subsets of data for training and testing. A first subset (e.g., a training subset) of the training data 804 can be used to build various models of prosthetic implants that have been tested using FEA. A second subset (e.g., a validation subset) of the training data 804 can then be used to validate the various models of prosthetic implants and/or any ML models that can be created or generated in accordance with the present disclosure. In some examples, the training data 804 can include actual patient data that includes fixation or fit data, bone preservation data, or other data obtained via C-rays, CT scans, or other imaging techniques. In one such example, one or more scans of one patient's anatomy can be received for a plurality of patients. The various input parameters can then be extracted from the one or more scans of the patient's anatomy for each of the plurality of patients, and subsequently saved as part of the training data 804. The ML model(s) can be exported for later use as needed.

The second stage 810 can include creating new implant designs. In one example, the second stage 810 can include creating one or more generic implant designs. The generic implant designs can be based on, for example, average bone density and load data of the bone of the patient. These generic implant designs can then be scaled to create a variety of different standardized sizes and shapes for a range of implants that can be selected by a surgeon to fit a population of different patients. In other examples, the second stage 810 can include creating patient-specific implant designs, such as via the fourth stage 512 (FIG. 8) and the fifth stage 514 (FIG. 8).

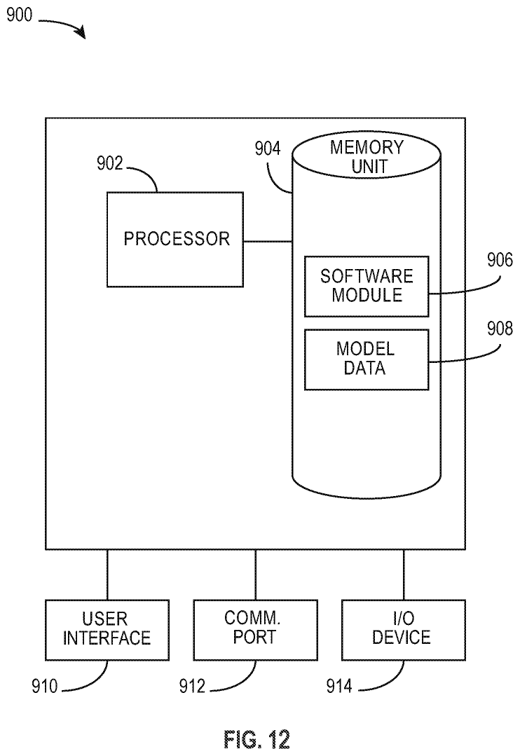

FIG. 12 illustrates an example schematic of computing device 900, in accordance with at least one example of this disclosure. As shown in FIG. 12, the computing device 900 can include a processor 902 and a memory unit 904. The memory unit 904 can include a software module 906 and model data 908. While executing on the processor 902, the software module 906 can perform processes for generating ML models, such as, but not limited to, generating one or more prosthetic implant designs, performing FEA or Musculoskeletal modeling on one or more generated designs, which can include, for example, one or more stages of the method 500, or one or more stages of the method 800, described above with respect to FIG. 8 and FIG. 11 above, respectively. The model data 908 can include, but is not limited to, training data, such as training data 804 (FIG. 9), design parameters used as inputs, databases, such as KNN databases, models generated using various prosthetic implant designs, or other data or information. The computing device 900 can also include a user interface 910, a communications port 912, and an input/output (I/O) device 914.

The user interface 910 can include any number of devices that allow a user to interface with the computing device 900. Some non-limiting examples of the user interface 910 can, but are not limited to, a keypad, a microphone, or a display (e.g., a touchscreen or otherwise). The communications port 912 can allow the computing device 900 to communicate with various information sources and devices, such as, but not limited to, remote computing devices, such as servers or other remote computers. For example, such remote computing devices can maintain data, such as model data, that can be retrieved by the computing device 900 using the communications port 912. Some non-limiting examples of the communications port 912 can include, but are not limited to, ethernet cards (e.g., wireless or wired), BLUETOOTH® transmitters and receivers, or near-field communications modules. The I/O device 914 can allow the computing device 900 to receive and output information. Some non-limiting examples of the I/O device 914 can include, a camera (e.g., still camera or a video camera), or fingerprint or other biometric scanners. For example, the I/O device 914 can allow the computing device 900 to directly receive patient data from a CT scanning device, an X-ray machine, or other imaging devices.

In view of all the above, the design processes of the present disclosure, such as including the method 800 and the method 500 (FIG. 8) can be implemented to design a patient specific implant or generic or standardized line of patient implants including lattice structures or porous metal sections, where the ML models can identify the best or otherwise optimal implant design for a given or individual patient. In various examples, the optimal implant tailored to best fit the trabecular bone volume, or any bone volume, of the patient, based on patient data such as scan information. Further, patient imaging data can be used to gain information on the patient bone quality and design the implant to profit from the denser bone regions. Also, the load may be tailored to the patient, based on general patient information.

While the above description of the design process of the present disclosure is generally discussed with reference to humeral and glenoid implants in the context of shoulder arthroplasties, the methods and systems disclosed herein can be used to design and manufacture a wide variety of prosthetic implants adapted for implantation within, or engagement with, various bones of the human body, such as but not limited to, stems for femoral, tibial, and humeral implants used in hip, knee and shoulder replacements, stems for humeral and ulnar implants used in elbow replacements, bases for glenoid or acetabular implants used in shoulder or hip replacements, as well as intramedullary nails, bone plates, or other implantable devices.

The foregoing systems and devices, etc. are merely illustrative of the components, interconnections, communications, functions, etc. that can be employed in carrying out examples in accordance with this disclosure. Different types and combinations of sensor or other portable electronics devices, computers including clients and servers, implants, and other systems and devices can be employed in examples according to this disclosure.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided.

Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein. In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure.

This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

### EXAMPLES

The following, non-limiting examples, detail certain aspects of the present subject matter to solve the challenges and provide the benefits discussed herein, among others.

Example 1 is a method for manufacturing an implant, the method comprising: receiving, at a computing device, patient specific data related to a bone; receiving, at the computing device, implant data related to the implant; determining, by the computing device, optimal design parameters for the implant using the patient specific data and a machine learning model; and exporting, by the computing device, the optimal design parameters.

In Example 2, the subject matter of Example 1 includes, where determining the optimal design parameters comprises determining regions of the bone with minimized stresses based on simulated loading of the implant.

In Example 3, the subject matter of Examples 1-2 includes, wherein determining the optimal design parameters comprises simulating a fit condition between the bone and the implant.

In Example 4, the subject matter of Examples 1-3 includes, wherein determining the optimal design parameters comprises simulating loading of the implant.

In Example 5, the subject matter of Example 4 includes, wherein simulating loading of the implant comprises applying loading and moments.

In Example 6, the subject matter of Examples 1-5 includes, wherein determining the optimal design parameters comprises optimizing a lattice structure of the implant at portions of the implant with minimal stresses.

In Example 7, the subject matter of Examples 1-6 includes, wherein determining the optimal design parameters comprises: changing a portion of the implant data representing a portion of the implant from a first value to a second value; and simulating a fit condition between the bone and the implant.

In Example 8, the subject matter of Example 7 includes, wherein the first value represents solid material and the second value represents porous material.

In Example 9, the subject matter of Examples 1-8 includes, wherein determining the optimal design parameters comprises determining a size of a placement fixture.

In Example 10, the subject matter of Examples 1-9 includes, where determining the optimal design parameters comprises determining at least one of a placement, a shape, and a size of at least one placement fixture.

In Example 11, the subject matter of Examples 1-10 includes, generating a plurality of designs for the implant, each of the plurality of designs including a different permutation of design parameters.

In Example 12, the subject matter of Examples 1-11 includes, wherein receiving the patient specific data comprises: receiving a scan of a patient's anatomy; and extracting design constraints for the implant from the scan.

In Example 13, the subject matter of Examples 1-12 includes, wherein exporting the optimal design parameters includes transmitting the optimal design parameters to an automated manufacturing device.

Example 14 is a system for manufacturing an implant, the system comprising: at least one processor; and at least one memory storing instructions that, when executed by the at least one processor, cause the at least one processor to perform actions comprising: receiving patient specific data related to a bone; receiving implant data related to the implant; determining optimal design parameters for the implant using the patient specific data and a machine learning model; and exporting the optimal design parameters.

In Example 15, the subject matter of Example 14 includes, where determining the optimal design parameters comprises determining regions of the bone with minimized stresses based on simulated loading of the implant.

In Example 16, the subject matter of Examples 14-15 includes, wherein determining the optimal design parameters comprises simulating a fit condition between the bone and the implant.

In Example 17, the subject matter of Examples 14-16 includes, wherein determining the optimal design parameters comprises simulating loading of the implant.

In Example 18, the subject matter of Example 17 includes, wherein simulating loading of the implant comprises applying loading and moments.

In Example 19, the subject matter of Examples 14-18 includes, wherein determining the optimal design parameters comprises optimizing a lattice structure of the implant at portions of the implant with minimal stresses.

In Example 20, the subject matter of Examples 14-19 includes, wherein determining the optimal design parameters comprises: changing a portion of the implant data representing a portion of the implant from a first value to a second value; and simulating a fit condition between the bone and the implant.

In Example 21, the subject matter of Example 20 includes, wherein the first value represents solid material and the second value represents porous material.

In Example 22, the subject matter of Examples 14-21 includes, wherein determining the optimal design parameters comprises determining a size of a placement fixture.

In Example 23, the subject matter of Examples 14-22 includes, where determining the optimal design parameters comprises determining at least one of a placement, a shape, and a size of at least one placement fixture.

In Example 24, the subject matter of Examples 14-23 includes, generating a plurality of designs for the implant, each of the plurality of designs including a different permutation of design parameters.

In Example 25, the subject matter of Examples 14-24 includes, wherein receiving the patient specific data comprises: receiving a scan of a patient's anatomy; and extracting design constraints for the implant from the scan.

In Example 26, the subject matter of Examples 14-25 includes, wherein exporting the optimal design parameters includes transmitting the optimal design parameters to an automated manufacturing device.

Example 27 is at least one machine-readable medium including instructions that, when executed by processing circuitry, cause the processing circuitry to perform operations to implement of any of Examples 1-26.

Example 28 is an apparatus comprising means to implement of any of Examples 1-26.

Example 29 is a system to implement of any of Examples 1-26.

Example 30 is a method to implement of any of Examples 1-26.

What is claimed is:

1. A method for manufacturing an implant, the method comprising:
receiving, at a computing device, patient specific data related to a bone;
receiving, at the computing device, implant data related to the implant;
determining, by the computing device, optimal design parameters for the implant using the patient specific data and a machine learning model; and
exporting, by the computing device, the optimal design parameters.

2. The method of claim 1, where determining the optimal design parameters comprises determining regions of the bone with minimized stresses based on simulated loading of the implant.

3. The method of claim 1, wherein determining the optimal design parameters comprises simulating a fit condition between the bone and the implant.

4. The method of claim 1, wherein determining the optimal design parameters comprises simulating loading of the implant.

5. The method of claim 4, wherein simulating loading of the implant comprises applying loading and moments.

6. The method of claim 1, wherein determining the optimal design parameters comprises optimizing a lattice structure of the implant at portions of the implant with minimal stresses.

7. The method of claim 1, wherein determining the optimal design parameters comprises:
changing a portion of the implant data representing a portion of the implant from a first value to a second value; and
simulating a fit condition between the bone and the implant.

8. The method of claim 7, wherein the first value represents solid material and the second value represents porous material.

9. The method of claim 1, wherein determining the optimal design parameters comprises determining a size of a placement fixture.

10. The method of claim 1, where determining the optimal design parameters comprises determining at least one of a placement, a shape, and a size of at least one placement fixture.

11. The method of claim 1, further comprising generating a plurality of designs for the implant, each of the plurality of designs including a different permutation of design parameters.

12. The method of claim 1, wherein receiving the patient specific data comprises:
receiving a scan of a patient's anatomy; and
extracting design constraints for the implant from the scan.

13. The method of claim 1, wherein exporting the optimal design parameters includes transmitting the optimal design parameters to an automated manufacturing device.

14. A system for manufacturing an implant, the system comprising:
at least one processor; and
at least one memory storing instructions that, when executed by the at least one processor, cause the at least one processor to perform actions comprising:
receiving patient specific data related to a bone;
receiving implant data related to the implant;
determining optimal design parameters for the implant using the patient specific data and a machine learning model; and
exporting the optimal design parameters.

15. The system of claim 14, where determining the optimal design parameters comprises determining regions of the bone with minimized stresses based on simulated loading of the implant.

16. The system of claim 14, wherein determining the optimal design parameters comprises simulating a fit condition between the bone and the implant.

17. The system of claim 14, wherein determining the optimal design parameters comprises simulating loading of the implant.

18. The system of claim 17, wherein simulating loading of the implant comprises applying loading and moments.

19. The system of claim 14, wherein determining the optimal design parameters comprises optimizing a lattice structure of the implant at portions of the implant with minimal stresses.

20. The system of claim 14, wherein determining the optimal design parameters comprises:
changing a portion of the implant data representing a portion of the implant from a first value to a second value; and
simulating a fit condition between the bone and the implant.

21. The system of claim 20, wherein the first value represents solid material and the second value represents porous material.

22. The system of claim 14, wherein determining the optimal design parameters comprises determining a size of a placement fixture.

23. The system of claim 14, where determining the optimal design parameters comprises determining at least one of a placement, a shape, and a size of at least one placement fixture.

24. The system of claim 14, further comprising generating a plurality of designs for the implant, each of the plurality of designs including a different permutation of design parameters.

25. The system of claim 14, wherein receiving the patient specific data comprises:
receiving a scan of a patient's anatomy; and
extracting design constraints for the implant from the scan.

26. The system of claim 14, wherein exporting the optimal design parameters includes transmitting the optimal design parameters to an automated manufacturing device.
